(19) United States
(12) Patent Application Publication
(10) Pub. No.: US 2004/0101842 A1
(43) Pub. Date: May 27, 2004

(54) Method to screen for improved meat characteristics in pigs

(76) Inventors: Christoper Simon Haley, Edinburgh (GB); Charlotte Anne Matlin, Aberdeen (GB)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/296,680

(22) PCT Filed: May 25, 2001
(86) PCT No.: PCT/GB01/02338

(30) Foreign Application Priority Data
May 27, 2000 (GB) 0012860.3

(51) Int. Cl. C12Q 1/68

(52) U.S. Cl. 435/6

(57) ABSTRACT

There is provided an assay to identify pigs having a genetic predisposition to musculature with improved meat quality characteristics. In the assay certain genetic markers which correlate to the meat quality traits of interest are used to determine the allelic variant(s) in the DNA sample under test. Preferred markers are: i) SW413, SW1482, SW439, S0005, SW904, or regions of chromosome 5 spanning therebetween; or ii) SWR68, S0024, SW827, SW727, SW539, or regions of chromosome 9 spanning therebetween; or iii) SW2094, SW2116 or regions of chromosome 9 spanning therebetween. From the genotypic data so generated pigs of the preferred genotype can be selected for slaughter or use in breeding programs. A kit for conducting the assay is also described.

The present invention relates to pigs having musculature with improved meat quality, and ways to identify them, including muscle fibre characteristics and genetic markers. In particular, the present invention provides an assay to screen pigs for improved meat quality characteristics such as tenderness, shear force and muscle fibre characteristics.

In the United Kingdom, elsewhere in Europe and increasingly throughout the world, pig producers are selecting breeds to use on their farms that are efficient producers of lean meat of high quality and thus provide the farmer with the maximum possible economic return.

‘White’ breeds of pig, like the ‘Large White’ and ‘Landrace’ especially those produced by pig breeding companies in the United Kingdom are characterised by having a good growth rate and producing carcases with a low subcutaneous and intermuscular fat level and thus a high lean content. These characteristics also lead to animals with a high feed conversion efficiency. Considerable progress in improving the lean meat content of these breeds of pig has been made in recent years in the United Kingdom.

There are reasons to believe that this long-term selection for lean content may have had the consequence of coincidentally selecting for pigs with a biological predisposition to poor meat quality. In particular, the lean meat may be increasingly predisposed to a problem known as Pale Soft Exudative meat (PSE), and may have eating quality problems such as toughness and dryness.

Another important world breed of pig is the ‘Duroc’. This is a North American breed of meat pig, red in colour and originating between 1822 and 1877 from ‘Old Duroc’ of New York and ‘Jersey Red’ of New Jersey. A breed society was formed in 1833 (Mason 1988). The ‘Duroc’ remains very popular in the United States and many were imported into Europe during the twentieth century.

Within Europe, especially the United Kingdom, the ‘Duroc’ is characterised as being of reasonable growth rate, but fatter and less efficient with regard to meat production than ‘Large White’ and ‘Landrace’. However, it has been shown a number of times to have meat of superior quality, especially colour and tenderness, than the “White” breeds (as defined above).

In Canada, Denmark, France and New Zealand, pigs produced from “White” hybrid mothers and ‘Duroc’ sires

have produced pigs with a tenderness advantage ranging from 10 to 17% over similar but ‘White’ sired pigs (Martel et al 1988; Barton-Gade 1989; Gandemer and Legault 1990 and Purchas et al 1990).

The interest in the ‘Duroc’ breed in the United Kingdom prompted the Meat and Livestock Commission to undertake what is probably the most comprehensive evaluation of the breed ever done. Conventional ‘White’ British commercial pigs (‘Large White’ sires crossed to ‘Large White’ cross ‘Landrace’ dams) containing zero percent ‘Duroc’ genes were compared with pigs containing 25, 50 or 75% ‘Duroc’ genes produced by various crosses (MLC 1992). Some results for 0% and 50% ‘Duroc’ pigs (ie. 100% and 50% “White” pigs) are presented in Table 1 and illustrate the relative merits of the two pig types.

TABLE 1

DUROC CONTENT

| | 0% | 50% |
|---|---|---|
| Daily live weight gain (g) | 806 | 803 |
| Feed conversion ratio | 2.70 | 2.83 |
| Lean tissue feed conversion ratio | 6.19 | 6.81 |
| P₂ fat depth (mm) | 9.3 | 10.9 |
| Lean % | 58.8 | 56.6 |
| PSE carcases (%) | 8.3 | 1.6 |
| Deep seated hair (% carcases) | 1.1 | 17.6 |
| Tenderness score* | 4.96 | 5.32 |
| Pork flavour* | | |
| In lean | 3.88 | 3.96 |
| In fat | 3.87 | 4.06 |
| Pork odour in fat* | 3.58 | 3.73 |

*sensory scores are on a 1-8 scale where higher scores indicate more tender, juicy etc. All results are for pigs fed ad-libitum but restrictedly fed pigs show similar results (MLC 1992).

Thus it can be seen that ‘Duroc’ cross pigs have good quality meat in comparison to ‘White’ pigs but this is obtained at the expense of being less efficient, fatter and having other carcase quality problems.

The difference between ‘White’ and ‘Duroc’ with regard to tenderness illustrates the existence of a genetic component for meat quality traits, that may equally exist between other breeds or within breeds or crosses. It is not a proof that the ‘Duroc’ always has better meat quality than ‘White’, the reverse may also be true on occasions.

Tenderness is a particularly important trait because, as described by Warkup et al (1995), previous experience of the product plays a major role in the consumer's decision to buy it again. Unlike attributes like the animal's welfare, residues and food hygiene (unless consumption results in illness), sensory attributes are actually experienced by the consumers. Studies quoted by Warkup et al (1995) indicate that tenderness is the most important attribute of meat.

The sensation of tenderness by a consumer can be assessed by a trained taste panel. Trained panels operating under strictly controlled conditions are able to detect smaller differences in tenderness and other meat quality traits than the consumer at large. Example 1 includes a description of a trained taste panel operated to assess meat quality attributes.

Tenderness of meat can also be measured instrumentally, and is then defined as the shear force. The force required to cut through a piece of meat is measured and can be expressed as the force at first yield, total work and maximum force or related traits. Example 1 includes a description of exemplary measurement of shear force traits.

Correlations between shear force and taste panel scores for tenderness (with low scores for tender meat and high scores for tough meat) vary from 0.27 to 0.78 (Stumpe 1989).

To date there is no clear explanation of what causes the meat quality differences between White breeds and Duroc. There is a widely held belief that the level of fat in the muscle (intramuscular) fat may be important (Bejerholm 1984) but there are contradictory views about the role of fatness and the ‘Duroc’ clearly differs from ‘White’ pigs in more respects than just fatness.

One of the observations made in our own earlier studies (MLC 1992) was that pigs containing ‘Duroc’ genes have a higher level of haem pigment. This observation and the higher levels of intramuscular fat are an indication of a higher oxidative capacity in the muscle.

Muscle (and hence meat) is made up of a variety of different muscle fibre cell types, which can be classified according to their contractile and metabolic nature. The two major classes of fibre type identified on the basis of their contractile nature (fast twitch and slow twitch) are subdivided into a number of subtypes based on their metabolic nature. Thus, according to one method of classification (see Peter et al 1972) muscle is shown to comprise slow-twitch oxidative (SO), fast-twitch glycolytic (FG), fast-twitch oxidative/glycolytic (FOG) and fast-twitch oxidative muscle fibre types. The proportions of the fibre types vary between muscles.

These fibre types are common to most muscles from most meat animals and typically show a random distribution throughout the tissue. However, in the pig the SO fibres are arranged with clusters or groups and are surrounded by fast twitch fibre types (Szentkuti and Cassens 1978). This association of muscle cells of similar metabolic types was described as forming “metabolic” clusters (Handel and Stickland 1987). The number of SO clusters is believed to be proportional to the number of primary fibres formed during myogenesis, the number of primary fibres being fixed in the pig foetus by 70 days gestation.

There is evidence of differences in the proportions of these different fibres among pig breeds (Iwamoto et al 1983; Ruusunen 1993). Differences in proportion of different fibre types have also been shown to occur among different pig breeds when fibre proportion is analysed for bundles of mixed fibre types (Skorjanc et al 1994). There has also been a tendency for breed crosses including ‘Duroc’ to have more SO and more FOG fibres (Uhrin et al 1986). This latter observation is entirely consistent with the proposed higher oxidative capacity as indicated by higher haem content.

The clearest breed difference in SO frequency was that seen by (Ruusunen 1993). These workers examined the fibre type composition of the Longissimus Dorsi of 38 pure ‘Hampshire’ (H), 52 ‘Finnish Landrace’ (L) or ‘Yorkshire’ (Y) sires cross onto (L×Y females), and 52H sires crossed onto (L×Y females) pigs. SO frequency was 15.3%, 11.5% and 11.6% respectively. The H had significantly more SO fibres than either cross. The fibre composition of the H cross animals more closely resembled the composition of the animals which did not contain H than the pure H animals. This confirms that breed differences for meat quality characteristics are not limited to comparisons including ‘Duroc’.

Results from recent studies of porcine longissimus muscle, presented in WO-A-98/15837 show:

1. That the percentage frequency of SO fibres and the proportional area of SO fibres per unit muscle is increased in the Duroc pig relative to the “White” pig;

2. That the number of SO fibres per cluster is increased in the Duroc pig relative to the “White” pig;

3. That m calpain is preferentially localised in the SO fibres of pigs. Therefore pigs with more SO fibres (eg Duroc) have more m calpain in the muscle as a whole. Thus the amount of m calpain is increased per unit muscle in the Duroc pig relative to the “White” pig;

4. That the amount of μ calpain per fibre is increased in the Duroc pig relative to the “White” pig;

It is well documented that post mortem storage of animal carcases at below ambient temperature, but above freezing, results in an improvement in meat tenderness. This increase in tenderness is due to the enzymatic breakdown of myofibrillar proteins and there is evidence that calpains are responsible for 90% of the tenderisation that occurs during post mortem storage (Taylor et al 1994). Calpains are intracellular, calcium activated/dependent thiol proteases present to some extent in most body tissues. However, their exact role in normal physiological conditions is still undefined. Several isoforms of calpain are known to occur in various body tissues of birds and animals. Two isoenzymes, μ calpain and m calpain, with different calcium requirements were originally isolated (Huston and Krebs 1968, Mellgren 1980). More recently tissue specific calpains have been isolated from skeletal muscle and stomach (Sorimachi et al 1989, Sorimachi et al 1993). It is the actions of μ calpain and m calpain that are thought to be involved in post mortem tenderisation of meat. In animal carcasses μ calpain is most active during the first 15 hours post slaughter whereafter its activity declines rapidly whilst the activity of m calpain is much more persistent. The activity of both μ and m isoforms of calpain is regulated by a natural inhibitor, calpastatin, which is also ubiquitously distributed in all body tissues.

Studies presented in WO-A-98/15837 have shown that m calpain is concentrated in the SO fibres of pig muscle. As Duroc meat has a greater proportion of SO fibres compared to meat from other breeds the corresponding increase in m calpain levels could account for the tenderness of Duroc meat.

It was also found that there is an overall increased amount of μ calpain per fibre in the muscles of Duroc pigs. An increased concentration of μ calpain per fibre could also explain the increased tenderness of Duroc meat.

Selection of animals with a genetic predisposition to better meat quality would be an attractive and cost-effective method to improve meat quality. The identification of animals of the desired genotype (genetic make up) requires some understanding of the nature of genetic variation and methods to detect it.

An animal's phenotype is the result of complex actions of the genes inherited from its parents and environmental factors. Most traits of agricultural importance in animal production are influenced by variation at several or many different genes. Usually individual genes do not have a large enough effect on their own to produce observable qualitative differences between individuals. More commonly, variation in several or many genes combines to produce continuous or quantitative variation between animals in traits such as growth rate and fatness.

Genome mapping can be used to identify the location of genes that influence variation in quantitative traits. The loci affecting quantitative traits are termed quantitative trait loci or QTLs.

The tools used to follow the inheritance in different chromosomal regions are genetic markers and these can be selected from the genome map to ensure coverage of the entire genome.

Maps showing distances between ordered loci can be built using recombination frequencies between pairs of loci or between multiple groups of loci.

For example, linkage maps of the porcine genome now contain substantial amounts of information and their status is constantly changing. Published linkage maps and linkage data are stored in the genome databases, for the pig this is PiGBASE/ARKdb-pig: URL=http://www.thearkdb.org.

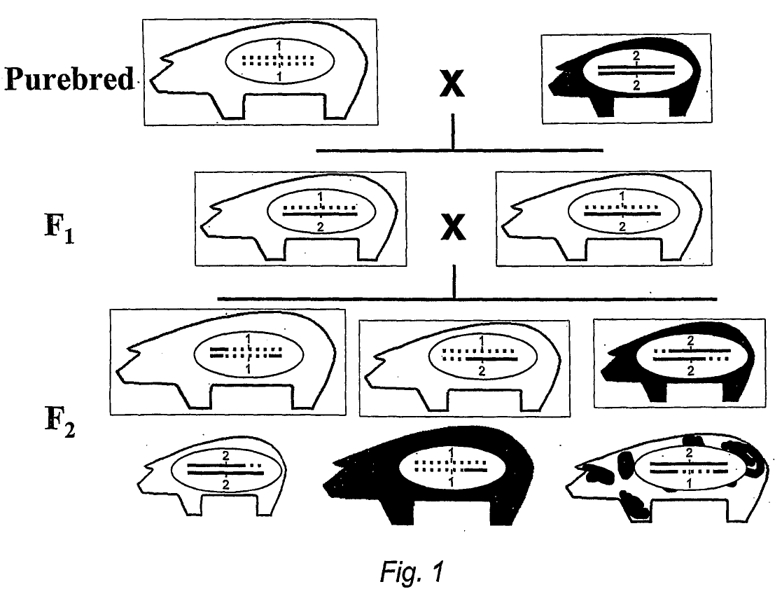

The basic principle of showing a gene or a region of the genome is associated with variation is illustrated in FIG. 1 for pigs. It consists of identifying a genetic marker and showing that its inheritance in a suitable pedigree is associated with variation in performance.

In a population such as that derived from the cross between two lines illustrated in FIG. 1, there may be an overall association between a particular marker allele and a particular allele at a quantitative trait locus (QTL). Linkage disequilibrium between a QTL and a marker leads to an overall association between the marker allele and the quantitative trait. In a random mating population, recombination over a number of generations will lead to the gradual decay in linkage disequilibrium between loci, with the rate of decay related to the distance between the loci.

Genome studies often analyse several or many different markers when looking for an effect on the phenotype. Thus, a number of effects may be significant by chance if the standard 5% significance level is used. Hence, it is recommended practise to use a more stringent significance level such that the overall chance of finding a significant result amongst all the markers tested is no more than 5% (see Lander and Kruglyak (1995) for a more detailed discussion of these points). This means that nominal significance levels at 0.01-0.001% or higher may be used in some studies. This in turn increases the sample size required for results to be significant at this level.

In genome scans for pigs where 19 chromosomes are tested and many positions within chromosomes, use of the nominal threshold is likely to lead to a number of false positive results reaching this significance threshold. Hence, QTL results are usually judged against a genome wide significance threshold (probability of a false positive for a single trait <0.05 in the entire genome, equivalent to an F value >9.0 for the pig genome) or the less stringent genome wide suggestive significance threshold (expect one false positive per trait in a whole genome scan, equivalent to F>5.0 approximately in the pig genome). See table below for further clarification:

Expected number of false positives in scan of:

| | F | Whole | One | Single |
|---|---|---|---|---|
| Threshold | value | genome | chromosome | Point |
| Nominal | >3.0 | ca. 5 | ca. 0.25 | 0.05 |
| Suggestive | >5.0 | 1.0 | 0.05 | 0.01 |
| Significant | >9.0 | 0.05 | <0.001 | <0.0001 |

The full power of the map and markers is employed in performing a genome scan for loci affecting traits of interest. The strength of this approach is that it has the potential to detect any loci with a large effect on a studied trait, whether or not their existence is known in advance. To implement this approach, markers which are spaced at intervals through the genome and which are polymorphic in the population being studied are selected from the map. The phenomenon of genetic linkage means that each marker can be used to follow the inheritance of a section of linked chromosome. Around 100-150 evenly spaced markers are needed to cover the whole genome and follow the inheritance of all sections. Thus maps of highly polymorphic markers are very valuable for this approach, as they allow selection of markers that provide this coverage and that are informative in the population of interest.

Thus the genome scan can both localise known genes of major effect and identify loci that were not known a priori. A significant amount of work is required to type sufficient animals for markers covering the entire genome. However, it is possible to design an experiment such that there is a high probability of detecting a gene of a defined effect on the phenotype wherever it is in the genome. More details on genome scans can be accessed in research publications, review articles and textbooks.

We have conducted such a genome scan for QTL contributing to variation in meat quality and its component traits, including muscle fibre characteristics.

The present invention is concerned with the use of genetic markers to identify animals with superior genes for meat quality traits.

The invention is founded upon the following novel observations:

1. Pig genetic markers SW413, SW1482, SW439, S0005 and SW904 or regions of chromosome 5 spanning therebetween are associated with shear force, muscle fibre characteristics and eating quality and related meat quality traits;

2. Pig genetic markers SWR68, S0024, SW827, SW727 and SW539 or regions of chromosome 9 spanning therebetween are associated with muscle fibre characteristics, shear force, tenderness and related meat quality traits;

3. Pig genetic markers SW2093 and SW2116 or regions of chromosome 9 spanning therebetween are associated with muscle fibre characteristics and related meat quality traits;

Note that the observed genetic effects are different from those found by Soumillion et al 1997 who established an association between meat fibres and the Myogenin gene, located at the middle of pig chromosome 9.

The specific markers referred to above detailed in the website http://www.thearkdb.org and brief details of these markers are also set out in Example 1.

Experimental details, including primer sequences for many of the genetic markers, can also be found on the USDA Meat Animal Research Centre, WWW site at http://sol.marc.usda.gov.

The present invention provides an assay to identify pigs with a genetic predisposition for improved meat quality, wherein said assay comprises:

a) obtaining a DNA sample from a test pig;

b) analysing the sample to determine the allelic variant(s) present at a genetic marker, wherein said marker is selected from:

i) SW413, SW1482, SW439, S0005, SW904 or regions of chromosome 5 spanning therebetween; or

ii) SWR68, S0024, SW827, SW727, SW539, or regions of chromosome 9 spanning therebetween; or

iii) SW2093, SW2116 or regions of chromosome 9 spanning therebetween; and

c) using the genotypic data from said marker(s) to select for pigs of the preferred genotype.

By “improved meat quality” or “high meat quality” we refer to animals which yield meat exhibiting desirable traits of tenderness and shear force.

For clarity it should be understood that the assays referred to herein may be conducted on individual animals or, for reasons of economy, may be conducted on pooled genetic samples for a group of animals.

In a yet further aspect, the present invention provides a method of identifying pigs which have a genetic disposition for improved meat quality, said method comprising:

a) obtaining a DNA sample from said pig;

b) assaying said DNA sample for a sequence identical with or complementary to the genetic markers identified above.

The animals identified by the assays referred to herein may be slaughtered to provide high quality meat and/or may also be selected for breeding programs.

Accordingly the present invention also provides a method of selecting pigs for use in breeding programs, said method comprising obtaining a DNA sample from a test pig and analysing said sample to determine the allelic variant(s) present at a genetic marker as described above, and using the genotypic data from said marker to select for pigs having the required genotype.

Although the study looked at the particular markers identified above, it is known to those skilled in the art that other genetic markers from within the QTL or the neighbouring portions of pig chromosome 5 or 9, or their homologues in other mammals (as appropriate) may be used instead, provided of course that the marker(s) selected are found to map within or close to the QTL for meat quality traits.

Thus, the present invention provides a method to identify pigs with a genetic predisposition for improved meat quality, wherein said method comprises:

a) obtaining DNA samples from a population of pigs;

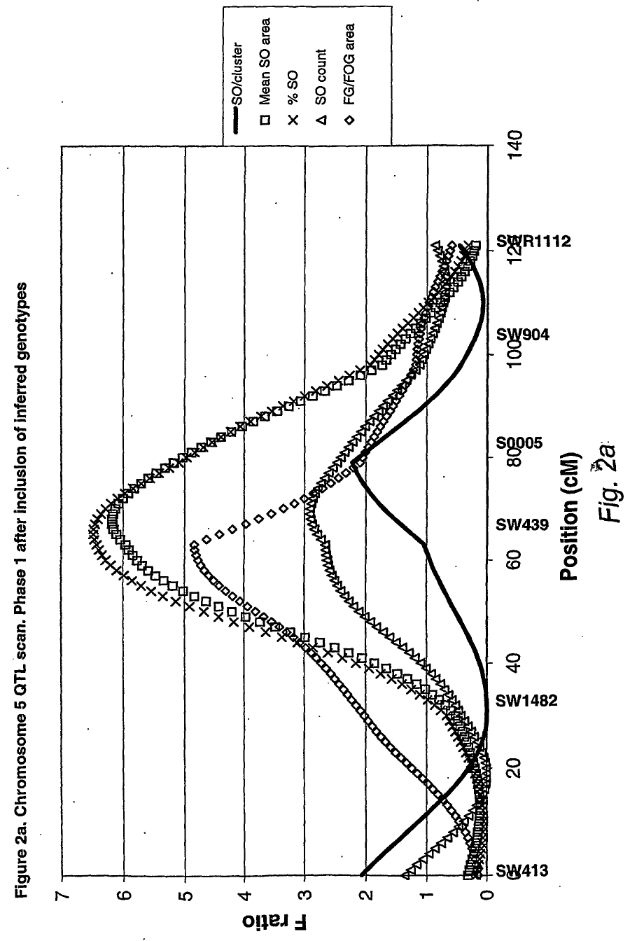

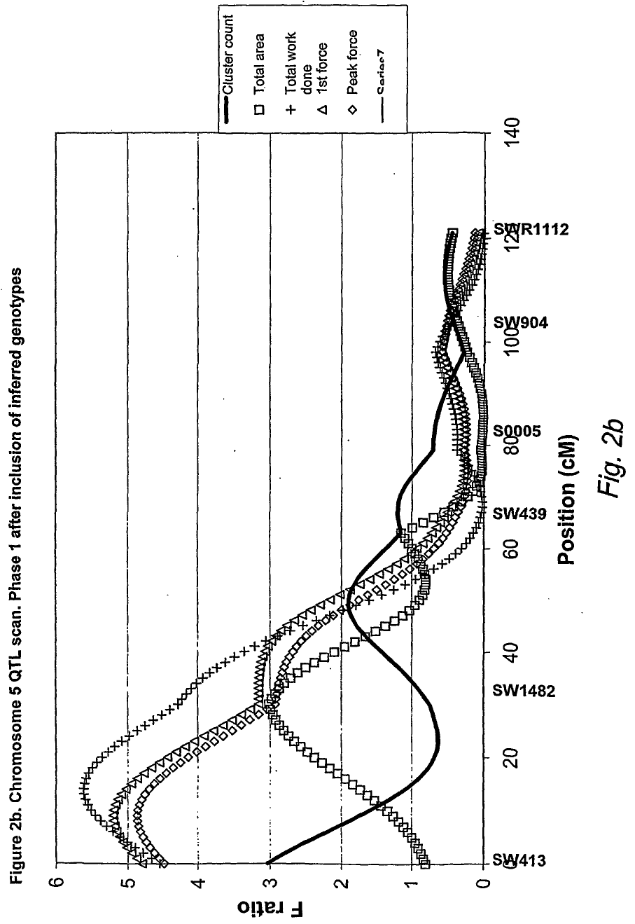

b) genotyping at least a sample of said population for pre-determined markers that map within or close to the QTL for meat quality traits defined herein (preferably on chromosomes 5 and 9, for example the specific markers referred to above or other markers located on either of chromosomes 5 and 9 where a high F ratio is indicated in any of FIGS. 2 to 5;

c) measuring meat quality traits for at least a sample of said population;

d) correlating the presence of allelic variants of said markers with said meat quality traits;

e) obtaining a DNA sample from a test pig;

f) analysing the sample to determine the allelic variant(s) present at a said selected genetic marker; and

g) using said marker results to select for pigs of the preferred genotype.

Steps a) and d) of the method described above are concerned with identifying markers which map within or close to the QTL for meat quality traits or with confirmation that the particular markers referred to are also relevant for the test population.

Preferably for pigs the markers are derived from SW413, SW1482, SW439, S0005, SW904, SWR68, S0024, SW827, SW727, SW539, SW2093 or SW2116. Other markers that map within or close to the QTL described herein can also be used. Particular mention may be made of any marker located on chromosome 5 in respect of shear force, or between or close to SW1482 and SW904 on chromosome 5 in respect of fibre traits, or between or close to SWR68 and SW2093 on chromosome 9 or between or close to SW2093 and SW2116 on chromosome 9. Preferably for other species, markers are derived from regions of the genome that are known to be homologous to the said regions on pig chromosome 5 and 9.

As can be seen in FIGS. 2 to 5 certain regions of chromosomes 5 and 9 correlate to high F ratios for specific traits connected to meat quality and markers in these regions may be of particular interest.

Optionally, a selection of markers that each allow the allelic variation at different QTL associated with meat quality to be predicted may be used in combination to achieve a more accurate prediction of meat quality predisposition. The present invention thus provides a kit to identify a pig having a genetic disposition for high meat quality said kit comprising at least three such genetic markers, preferably selected from the specific markers recited above, having the ability to identify specific allelic variant(s) at three separate QTL indicative of meat quality.

The animals shown to have marker genotypes or predicted QTL genotypes indicative of an improved meat quality predisposition, or the close relatives of such animals, can be used as breeding stock or for meat production.

In a further aspect the present invention provides a method of determining the genetic predisposition of a pig to yield meat of improved meat quality, said method comprising detecting genes located between the following pairs of markers:

i) SW413 and SW904 on chromosome 5;

ii) SWR68 and SW539 on chromosome 9; and

iii) SW2093 and SW2116 on chromosome 9;

wherein said genes are characterised by having allelic variant(s) which can influence meat quality or its component traits, or which are associated with variation in meat quality or its component traits.

Although the genetic markers used in this study are microsatellites the assay is not limited to the use of any particular technology or type of genetic marker. Any method for detecting DNA variation at specific chromosomal locations can be used to develop genetic markers that could be used for monitoring the inheritance of particular chromosomal segments or loci. It is clear to those skilled in the art that genetic markers, which map close to or within the QTL for muscle characteristics/meat quality traits defined herein, could be used in the assay for predicting on individual's predisposition for meat quality traits independent of the technology used to develop or genotype the marker. Thus, the assay is not limited to any particular type of genetic marker or genotyping technology, current or as yet undeveloped. Other genetic marker types and technologies include, but are not limited to, restriction fragment length polymorphisms (RFLPs), single strand conformational polymorphisms (SSCP), double strand conformational polymorphisms, single nucleotide polymorphisms (SNPs), AFLP™ (amplified fragment length polymorphisms), DNA chips, variable number of tandem repeats (VNTRs, minisatellites), random amplified polymorphic DNA (RAPDs), heteroduplex analyses, and allele-specific oligonucleotides (ASOs). Some DNA variation can be detected by assaying the variation in RNA transcripts or proteins. Thus, genetic marker technology for the purposes of the assay is not limited to direct measures of DNA variation.

Examples of markers that map to the muscle characteristics and meat quality QTL on pig chromosomes 5 (SSC5) and 9 (SSC9) include, but are not limited to, (marker type and chromosome are shown in parentheses) ACO2 (SSCP, SSC5); DAGK1, IGF1, IFNG (microsatellites, SSC5); MUC (RFLP, SSC5); PLP1 (protein variants, SSC5); EAE, EAK (erythorcyte antigen variants, SSC9); PPP2R1A, TYR, DLD (RFLPs, SSC9); MYOG (PCR-RFLP, SSC9); APOA1 (microsatellite, SSC9). Details of genetic marker technology can be accessed in primary research publications, review articles, textbooks and laboratory manuals.

Genes that map to the QTL regions identified on chromosomes 5 or 9 can be considered candidates for the genes determining the observed effects on meat quality traits. The basis of the candidature of these genes is their chromosomal locations. Hence, these genes are ‘positional’ candidate genes. Genes whose map location in pigs is currently unknown but which can be predicted to map to the QTL regions on chromosome 5 or 9 from knowledge of the map location of homologous genes in humans, mice and other species can be considered as ‘comparative positional’ candidates for the genes determining the observed meat quality traits.

Positional and comparative positional candidate genes determining functions that may contribute to the observed meat quality traits include, but are not limited to, the genes encoding: myogenic factor (MYF5); myogenic factor 6 (MYF6); collagen type II, alpha 1 (COL2A1); insulin-like growth factor 1 (IGF1); myosin phosphatase, target subunit 1 (MYPT1); myosin-binding protein C, slow-type (MYPC1); Wnt inhibitory factor 1 (WIF1); growth differentiation factor 11 (GDF11) and myogenin (MYOG). To those skilled in the art the isolation of the pig homologues of such candidate genes and the subsequent search for causal genetic variation in the candidate gene(s) is straightforward.

In the assay of the present invention, the genomic DNA will be detected from a sample of tissue donated from the pig, but the exact tissue forming the sample is not critical as long as it contains genomic DNA. Examples include (but are not limited to) body fluids such as blood, semen (sperm), ascites and urine; tissue and cells such as liver tissue, muscle, skin, hair follicles, ear, tail, fat and testicular tissue. The genomic DNA to be analysed can be prepared by extracting and purifying the DNA from such samples according to standard laboratory procedures.

The method may be conducted in vitro or in vivo using a sample from a living animal or post mortem following the death of the animal being tested. If the assay is conducted post mortem, the information obtained may be also of use for the siblings, parents or other close relatives of the animal.

The QTL for meat quality traits disclosed herein will allow the isolation and characterisation of the trait-genes themselves in pigs, since the positioning of the QTL enables a search for linkage to the genes responsible for the trait. Once these trait genes are located the option to manipulate the trait genes by transgenesis or to develop a further assay arises and forms part of the present invention.

Various genes and/or controlling sequences may be involved, especially the genes controlling the calpain/calpastatin system.

The invention will now be described with reference to the following, non limiting, examples and figures in which:

FIG. 1 depicts a three-generation pig pedigree produced by crossing divergent purebred lines of pigs to produce F₁ and F₂ generations. We focus on one small part of a single chromosome that carries a genetic marker with alternative alleles 1 and 2. The animals can be genotyped for this marker and the inheritance of alternative alleles can be followed through the pedigree. In the F₂ animals, both the marker and genes controlling the size differences between the breeds segregate. The marker acts as a signpost to show from which breed linked sections of chromosome are inherited. In this example the size of F₂ animals is associated with the marker genotype (animals with the 11 genotype are large, those with 22 are small). Hence a gene or genes for size is found in the region of chromosome inherited with the marker.

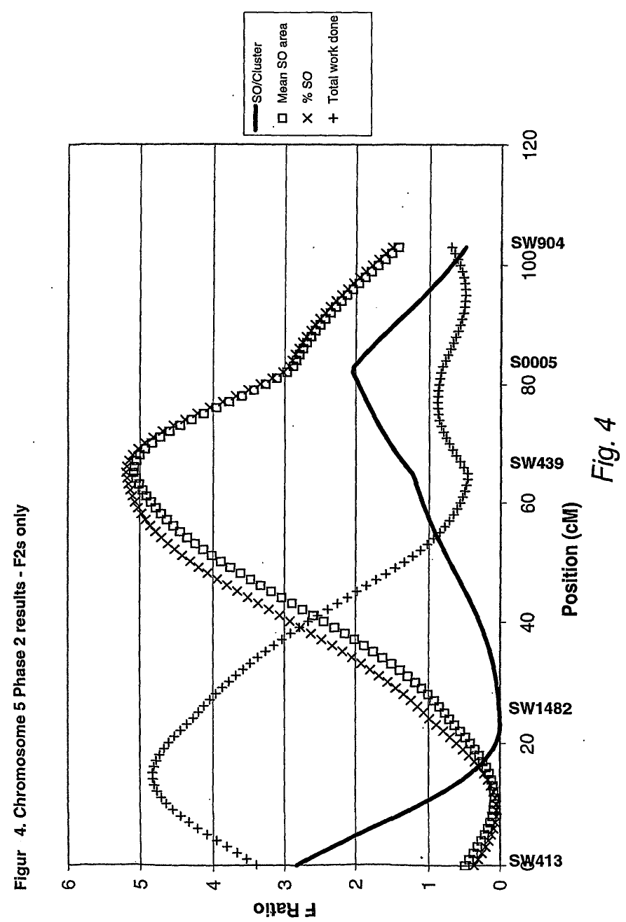

FIGS. 2 and 4 are graphs plotting the F value against position (cM) on pig chromosome 5 for different meat quality related traits.

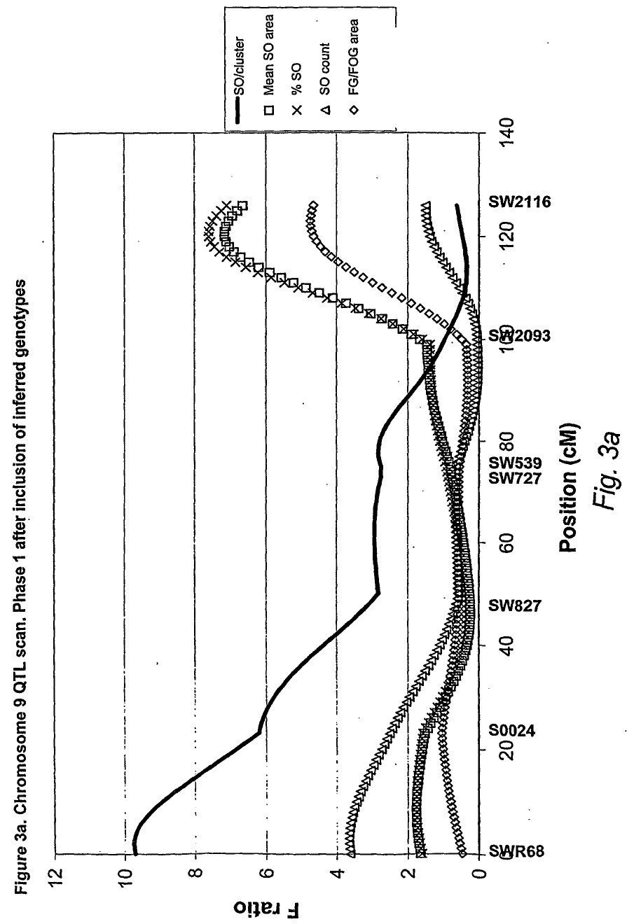

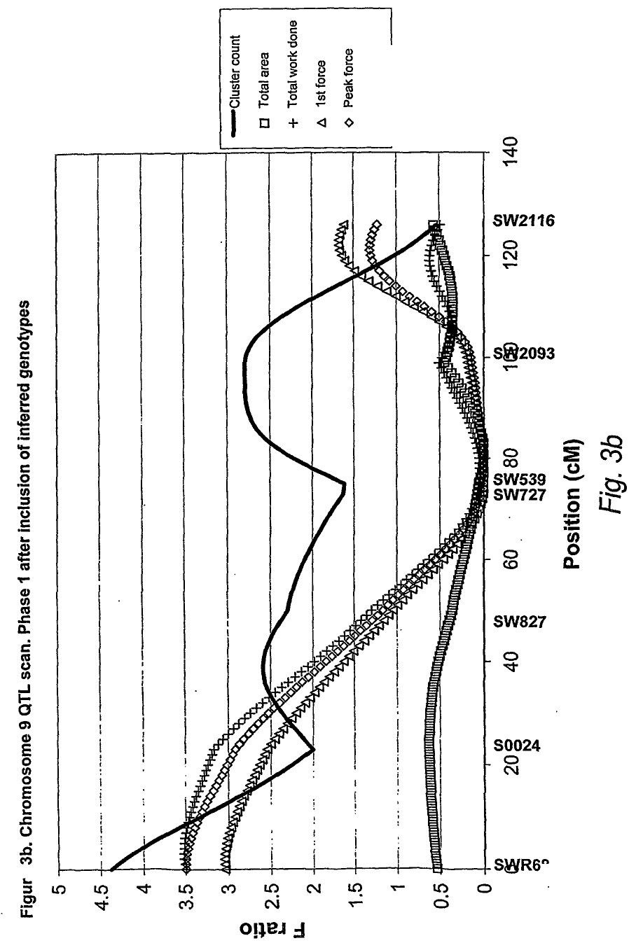

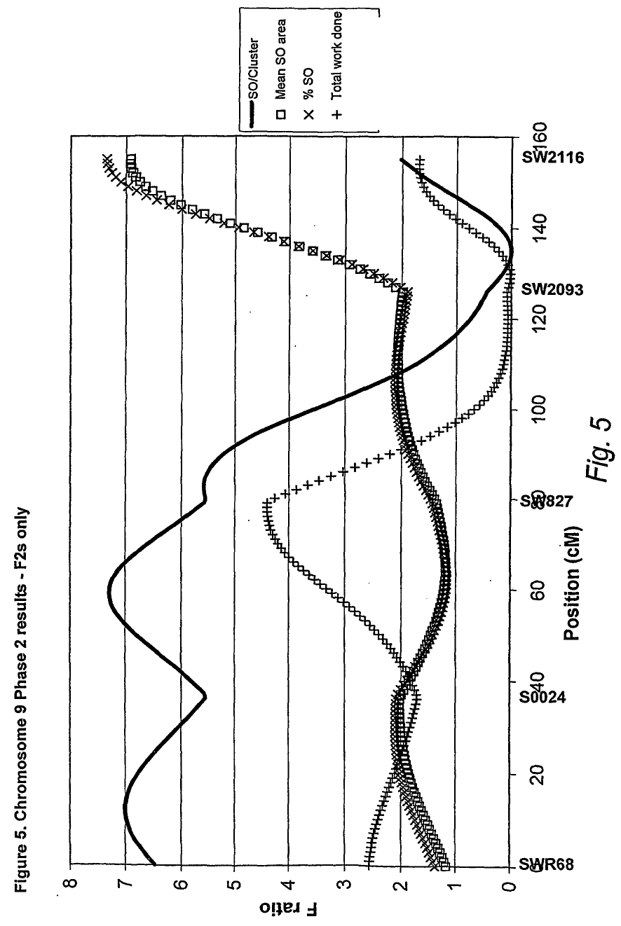

FIGS. 3 and 5 are graphs plotting the F value against position (cM) on pig chromosome 9 for different meat quality related traits.

### EXAMPLE 1

QTL Analysis

QTL mapping pedigrees were established in the form of three-generation families in which grandparents from genetically divergent breeds were crossed to produce the parental (F₁) generation which were subsequently intercrossed. The founder grandparental breeds were the Duroc and the European Large White (Yorkshire). About 120 F₂ animals were produced in these Large White/Duroc pedigrees.

Blood or tissue samples were taken from most grandparental, F₁ parental and F₂ pigs and these were used to prepare DNA.

Taste Panel, Shear Force and Fibre Traits

The phenotype markers were:

i) taste panel assessment of tenderness;

ii) taste panel assessment of overall acceptability;

iii) taste panel assessment of juiciness, pork flavour, abnormal flavour and boar flavour;

iv) shear force measurements as force at first yield, total work and maximum force;

v) muscle fibre characteristics traits as described below.

Tenderness, overall acceptability and the other taste traits (i to iii) were measured by the trained taste panel at the Meat and Livestock Commission. Two samples of meat for each animal were assessed in separate sessions by a trained sensory panel. There was a total of 365 sessions. At each panel session, meat samples from eight animals were analysed. Each of six panellists at that session was then given a separate sample of loin chop of each of the eight animals. Each panellist gave each animal a score for five attributes, on a scale of 1-24 (the higher the better) by marking a prepared form. The sample was assessed by mouth for juiciness, tenderness, pork flavour, abnormal flavour and boar flavour. Finally, a score was given for overall acceptability.

Each session and panellist involved in the trial had a unique number. The scores awarded by the panellists were analysed using the restricted maximum likelihood in a model fitting session number, panellist and individual animal number. Fitted values for each attribute for each individual were saved from these analyses and stored on a database for use in the QTL analyses.

For shear force measures (iv) the following protocol was used:

1) A 120 mm section of forequarter loin was removed anterior to the last rib.

2) After the removal from the carcase, joints were de-boned and de-rinded, labelled with the appropriate control number and vacuum-packed.

3) Samples were aged for seven days

4) In order to ensure uniform rapid freezing, samples were first placed in a blast-freezer before being transferred to the main cold store for storage at −30° C.

5) On removal from the cold store, samples were placed in the chiller at +3° C. for a period of 72 hours. Joints were placed on racks, avoiding overlap in order to facilitate consistency of thaw.

6) At 72 hours, the internal temperature of each joint was checked and only when all samples had internal temperatures of between 2 and 5° C. would cooking commence. After reaching the required temperature, each sample was re-vacuum packed and immediately taken to the Sensory Laboratory for cooking to commence.

7) Samples were placed in the water bath when the water temperature had reached 80° C. Each sample was cooked within its individual vacuum pack. One sample was used to monitor internal temperatures. This sample was cooked until the internal temperature reached 80° C., all samples were then cooked for a further 10 minutes.

8) After completion of cooking, samples were transferred to an iced water bath for one hour. Water was replaced every 15 minutes.

9) After the one hour period, all samples were taken to the cutting room chiller and stored overnight at +3° C. They were laid on racks in order to ensure good air circulation.

10) The following day, ten replicate samples, each measuring 10 mm×10 mm×30 mm were removed from each sample, cutting each replicate along the direction of the fibres.

11) Replicates that had obvious tissue defects or did otherwise not represent a sample were discarded. If insufficient meat was available to replace these samples, then a lesser number than 10 was measured. Samples and replicates were kept covered and refrigerated between 2° C. and 5° C. until they were sheared.

12) The instrument used was a TA.XT2i Texture Analyser (Stable Micro Systems, England).

13) A Volodkevich (Stable Micro Systems, England) bite jaw was fitted.

14) The jaw was calibrated at 1.7 mm/s and travelled 8 mm into the sample.

15) The following were recorded on each replica:—

Force at first yield

Total work

Maximum force

Fibre typing fibre traits (v) were determined as follows:

Pigs were slaughtered when the mean litter live weight reached 90 kg.

Loin samples were removed for histochemical and DNA analysis 48 hours after slaughter.

The histochemical analysis of the muscle samples was carried out on approximately 1 cm² blocks cut from the centre of the longissimus dorsi muscle. Care was taken to ensure that the same area was sampled from each of the chops. These cubes of muscle were orientated for transverse sectioning, mounted on a piece of cork with optimal cutting temperature compound (OCT), covered with more OCT and with unperfumed talcum powder and frozen in liquid nitrogen with constant agitation. Twelve blocks were taken from each chop and once frozen, were stored in aluminium tins submerged in liquid nitrogen. Throughout the period of the study the blocks were maintained in the liquid phase of the nitrogen dewar to limit any freeze drying. The tins were removed from the liquid nitrogen storage and placed in the cryostat at −20° C. 2 hours before sectioning. Serial transverse sections were cut at 10 μm using a Frigocut 2800 cryostat with motor driven cutting stroke to reduce variation in section thickness.

The sections were allowed to air dry at ambient temperature for 2 hours and then frozen overnight for staining the following day.

The characterisation of fibre typing adopted in this study is based upon the reaction of individual fibres to a minimum of three stains. The stains used were chosen to demonstrate the activities of Ca²⁺ activated myofibrillar adenosine triphosphatase (ATPase), nicotinamide adenine dinucleotide diaphorase (NADH), and α-glycerophosphate dehydrogenase (GPOX), which then allowed the characterisation of the fibres based on their contractile and metabolic activities as follows and as illustrated in Table 2; ATPase—contractile activity (fast or slow twitch); NADH—oxidative activity; GPOX—glycolytic activity.

TABLE 2
The histochemical basis of characterisation
of muscle fibre types in pig meat.

| FIBRE | STAIN | | |
|---|---|---|---|
| TYPE | ATPASE | NADH | GPOX |
| FOG | ++(+) | +++ | +++ |
| FG | +++ | + | +++ |
| SO | + | +++ | + |

Quantification of Fibre Type and Size

Quantitative assessments of fibre type and size were made from the stained muscle preparations using a Torch computer based image analysis system (Vision Dynamics, Hemel Hempstead, Herts). Measurements of fibre size were made on the sections reacted to demonstrate the activity of ATPase. For each animal, fibre size estimation was carried out on eight blocks with two fields per block being analysed.

The ATPase stained sections were examined under a light microscope fitted with a Sony video camera, the output of which was applied to the image handling software of the Torch computer. The use of the ATPase stain generates an image in which three fibre types can be distinguished based on their grey levels. Fibre type was confirmed through examination of printed images of the NADH and GPOX stains to give information on the metabolic character of each fibre. The three fibre types were analysed separately, and thresholding was altered to detect all fibres of the same type. Where adjacent fibres were thresholded and detected as a single unit, manual editing operations were undertaken to separate the fibres through the use of a superimposed ‘live’ camera image to visualise the sarcolemmal membranes accurately. The data for size, frequency and percentage area was computed for each animal. Approximately 1600 fibres were analysed for each pig.

DNA samples were shipped to GeneSeek Inc (Lincoln, Nebr. USA) for genotyping. Marker alleles were amplified by PCR and scored following electrophoresis using infrared fluorescent technology. Markers were amplified using either 1) end-labelled forward primers, or 2) M13-tailed forward primers. Labelled forward primers were synthesised by LI-COR (Lincoln, Nebr. USA), while M13-tailed forward primers and all reverse primers were synthesised by Research Genetics (Huntsville, Ala. USA).

End-labeled reactions used 25 ng genomic DNA, 200 μM each DNTP, 0.15 picomol of labeled forward primer (either IR700 or IR800; LI-COR), 1 picomol of unlabeled reverse primer, 0.5 U Taq-Gold polymerase with supplied MgCl₂-free buffer (Perkin-Elmer; Foster City, Calif. USA), and 2.5 mM MgCl₂. M13-tailed reactions were the same except that 0.3 picomol of each primer were used. Each forward primer had a 19-bp 5′ tail consisting of M13 sequence, and each PCR included 0.3 picomol of a fluorescently labelled 19-bp M13 primer (either IR700 or IR800). Amplification began with an initial denaturation at 95° C. for 5 minutes, followed by “touchdown” PCR with annealing temperatures beginning at 68° C. and decreasing by 2° C. per cycle through to 54° C. A total of 33 cycles was performed at an annealing temperature of 54° C. PCR ended with a 7 minutes extension period at 72° C. PCR products were denatured at 95° C. prior to electrophoresis (1500V, 50 mA,

50W, 45° C.) in 7.0% denaturing polyacrylamide gels in LI-COR (Model 4200 IR2) sequencers.

Alleles were scored based on size relative to known DNA size standards. Genotyping results were stored in Excel files and delivered to the Roslin Institute as e-mail attachments and loaded into the resspecies database (http://www.resSpecies.org) at Roslin.

Details of the pedigree structure, dates of birth, sex and growth rate, carcase and slaughter characteristics, sensory and shear force evaluations and muscle fibre characteristics were loaded into the resspecies database (http://www.resSpecies.org) at Roslin Institute from Excel spreadsheets provided by the Rowett Research Institute.

The collated data on traits and marker genotypes were analysed to scan the genome for the presence of QTL influencing the traits of interest. The animals were genotyped for the genetic markers listed in Table 3. The markers were chosen to provide a reasonable spread over the whole of the genome.

TABLE 3
Markers used for genome scan.

| Marker | Chromosome | Position |
|---|---|---|
| SW1515 | 1 | 16 |
| SW1417 | 1 | 44 |
| SW1430 | 1 | 59 |
| S0331 | 1 | 73 |
| SW974 | 1 | 103 |
| SW2512 | 1 | 144 |
| SWC9 | 2 | 1 |
| SW575 | 2 | 32 |
| SW1026 | 2 | 61 |
| SWR2157 | 2 | 89 |
| S0036 | 2 | 132 |
| SW2429 | 3 | 17 |
| S0206 | 3 | 42 |
| SW902 | 3 | 58 |
| SW142 | 3 | 81 |
| SW349 | 3 | 113 |
| SW2404 | 4 | 0 |
| S0301 | 4 | 27 |
| S0175 | 4 | 56 |
| SW512 | 4 | 81 |
| SW445 | 4 | 106 |
| SW1461 | 4 | 120 |
| SW413 | 5 | 9 |
| SW1482 | 5 | 39 |
| SW439 | 5 | 72 |
| S0005 | 5 | 88 |
| SW904 | 5 | 107 |
| SWR1112 | 5 | 130 |
| SW2535 | 6 | 18 |
| SW1038 | 6 | 47 |
| DG87 | 6 | 63 |
| SW709 | 6 | 89 |
| S0121 | 6 | 116 |
| DG93 | 6 | 122 |
| SW2419 | 6 | 161 |
| S0025 | 7 | 4 |
| SW2155 | 7 | 33 |
| TNFB | 7 | 58 |
| SWR1928 | 7 | 79 |
| SW252 | 7 | 99 |
| S0101 | 7 | 135 |
| SW764 | 7 | 156 |
| S0353 | 8 | 12 |
| SWR1101 | 8 | 38 |
| S0086 | 8 | 62 |
| SW2160 | 8 | 80 |
| SW790 | 8 | 108 |
| S0178 | 8 | 128 |
| SWR68 | 9 | 4 |
| S0024 | 9 | 27 |
| SW827 | 9 | 54 |
| SW727 | 9 | 77 |
| SW539 | 9 | 79 |
| SW2093 | 9 | 103 |
| SW2116 | 9 | 130 |
| SWR136 | 10 | 7 |
| SW497 | 10 | 39 |
| SWR198 | 10 | 65 |
| SW1991 | 10 | 80 |
| SW1626 | 10 | 104 |
| SW2067 | 10 | 124 |
| SW1632 | 11 | 17 |
| S0071 | 11 | 50 |
| SW435 | 11 | 59 |
| SW13 | 11 | 86 |
| S0229 | 12 | 20 |
| SW1307 | 12 | 40 |
| SW874 | 12 | 65 |
| S0090 | 12 | 80 |
| SW2180 | 12 | 105 |
| SWR1941 | 13 | 14 |
| SW344 | 13 | 36 |
| S0068 | 13 | 62 |
| SW1386 | 13 | 77 |
| SW1056 | 13 | 96 |
| SW2097 | 13 | 121 |
| SW857 | 14 | 8 |
| SW1027 | 14 | 22 |
| SWR84 | 14 | 52 |
| SW761 | 14 | 76 |
| SWC27 | 14 | 112 |
| SW1416 | 15 | 13 |
| chr1-4 | 15 | 29 |
| SW964 | 15 | 51 |
| SW1683 | 15 | 79 |
| SW1983 | 15 | 102 |
| SWR312 | 15 | 120 |
| SW813 | 16 | 6 |
| SW2411 | 16 | 17 |
| SW81 | 16 | 40 |
| SW2517 | 16 | 56 |
| S0105 | 16 | 93 |
| SW335 | 17 | 0 |
| S0296 | 17 | 32 |
| S0359 | 17 | 68 |
| S0332 | 17 | 89 |
| SW1023 | 18 | 5 |
| SW1984 | 18 | 30 |
| S0177 | 18 | 55 |
| SW949 | X | 0 |
| SW980 | X | 12 |
| SW2126 | X | 35 |
| SW1943 | X | 87 |
| SW1608 | X | 102 |
| SW2588 | X | 128 |

Linkage maps of each pig chromosome were developed using Cri-Map version 2.4 (Green et al 1990). The linkage map positions for the markers on chromosomes 5 and 9 are presented in Table 3. The trait data and linkage maps were analysed by the least squares approach as described by Haley et al, 1994. All chromosomes were tested in this way (using appropriate markers for the chromosome under test), but the most significant correlation was found for meat quality with the markers on chromosomes 5 and 9.

Other more minor effects are given below in Table 4.

TABLE 4

| Chromosome | Trait |
|---|---|
| 3 | Total area (FG + FOG) |
| 7 | First force, peak force, total work, |
| | SO count, SO/cluster |

Analyses

All QTL analyses were performed by least squares. The assumption underlying these analyses is that QTL of major (i.e. detectable) effects were fixed for alternative alleles in the Duroc and Large White breeds that went into the study.

The models included fixed effects and any key covariates. Sex was always included as was either year or slaughter data as a fixed effect.

Results

The significant results for chromosomes 5 and 9 are set out in Table 5.

TABLE 5
Genome scan results by chromosome

| | | | F | % var | % var | Trait | | | | | Dominance |
|---|---|---|---|---|---|---|---|---|---|---|---|
| Trait | Chrom. | Position | ratio | 1 | 2 | s.d. | a | s.e. | d | s.e. | ratio |
| Clusters | 5 | 0 | 3.04 | 3.58 | 6.9 | 3.27E−01 | −6.19E−02 | 4.55E−02 | 1.48E−01 | 7.45E−02 | −2.39 |
| 1st force | 5 | 9 | 5.21 | 7.49 | 19.81 | 5.54E+02 | −2.22E+02 | 8.68E+01 | −3.81E+02 | 1.69E+02 | 1.72 |
| Peak force | 5 | 9 | 4.87 | 6.92 | 18.28 | 5.53E+02 | −2.20E+02 | 8.69E+01 | −3.56E+02 | 1.69E+02 | 1.62 |
| Total work | 5 | 14 | 5.62 | 8.16 | 20.34 | 1.06E+03 | −5.25E+02 | 1.71E+02 | −6.01E+02 | 3.45E+02 | 1.14 |
| done | | | | | | | | | | | |
| (shear) | | | | | | | | | | | |
| Total area | 5 | 30 | 3.01 | 3.53 | 6.98 | 6.23E+03 | 1.49E+03 | 9.40E+02 | −2.52E+03 | 1.62E+03 | −1.69 |
| (FG + | | | | | | | | | | | |
| FOG + | | | | | | | | | | | |
| SO) | | | | | | | | | | | |
| FG/FOG | 5 | 63 | 4.85 | 6.54 | 13.33 | 8.56E+03 | 3.50E+03 | 1.26E+03 | 3.81E+03 | 2.16E+03 | 1.09 |
| FG/FOG % | 5 | 65 | 6.48 | 9.06 | 15.84 | 2.20E+00 | 1.14E+00 | 3.25E−01 | 6.89E−01 | 5.79E−01 | 0.60 |
| SO % | 5 | 65 | 6.48 | 9.06 | 15.84 | 2.20E+00 | −1.14E+00 | 3.25E−01 | −6.89E−01 | 5.79E−01 | 0.60 |
| SO area | 5 | 68 | 6.17 | 8.6 | 14.69 | 5.40E+03 | −2.78E+03 | 8.07E+02 | −1.29E+03 | 1.50E+03 | 0.46 |
| Boar | 5 | 69 | 4.69 | 6.29 | 15.59 | 6.48E−01 | −2.06E−01 | 9.78E−02 | −4.20E−01 | 1.83E−01 | 2.04 |
| flavour | | | | | | | | | | | |
| (Adj.) | | | | | | | | | | | |
| PH 45 | 5 | 79 | 7.1 | 9.99 | 14.4 | 2.26E+01 | −5.14E+00 | 2.92E+00 | −1.55E+01 | 4.63E+00 | 3.02 |
| minutes | | | | | | | | | | | |
| Overall | 5 | 98 | 3.49 | 4.33 | 9.78 | 1.80E+00 | −6.30E−01 | 2.69E−01 | −6.91E−01 | 5.0E−01 | 1.10 |
| accepta- | | | | | | | | | | | |
| bility | | | | | | | | | | | |
| (Adj.) | | | | | | | | | | | |
| Juiciness | 5 | 98 | 4.97 | 6.73 | 12.47 | 1.96E+00 | −8.63E−01 | 2.89E−01 | −6.55E−01 | 5.38E−01 | 0.76 |
| (adj.) | | | | | | | | | | | |
| Pork | 5 | 111 | 4.34 | 5.72 | 17.57 | 1.37E+00 | −6.16E−01 | 2.34E−01 | −7.53E−01 | 4.83E−01 | 1.22 |
| flavour | | | | | | | | | | | |
| (Adj.) | | | | | | | | | | | |
| Abnormal | 5 | 120 | 4.09 | 5.32 | 18.7 | 8.76E−01 | −1.44E−01 | 1.43E−01 | 7.30E−01 | 2.74E−01 | −5.07 |
| flavour | | | | | | | | | | | |
| (Adj.) | | | | | | | | | | | |
| Clusters | 9 | 0 | 4.37 | 5.78 | 62.71 | 3.27E−01 | 6.47E−03 | 7.27E−02 | −5.18E−01 | 1.75E−01 | −80.06 |
| Hue | 9 | 0 | 4.19 | 5.48 | 49.17 | 3.13E+00 | 1.11E+00 | 6.96E−01 | −4.10E+00 | 1.68E+00 | −3.69 |
| Light | 9 | 0 | 4.58 | 6.11 | 63.05 | 1.90E+00 | 3.32E−01 | 4.21E−01 | −2.98E+00 | 1.02E+00 | −8.98 |
| Peak | 9 | 1 | 3.5 | 4.58 | 19.3 | 5.53E+02 | 3.42E+02 | 1.30E+02 | 4.38E+01 | 3.02E+02 | 0.13 |
| force | | | | | | | | | | | |
| 1st force | 9 | 2 | 3.05 | 3.79 | 16.65 | 5.54E+02 | 3.20E+02 | 1.30E+02 | 4.88E+00 | 3.03E+02 | 0.02 |
| SO/clust | 9 | 2 | 9.74 | 13.71 | 118.12 | 8.29E−01 | −2.52E−01 | 1.74E−01 | 1.77E+00 | 4.23E−01 | −7.02 |
| SO count | 9 | 3 | 3.66 | 4.61 | 39.31 | 1.97E+00 | −7.09E−01 | 4.30E−01 | 2.25E+00 | 1.05E+00 | −3.17 |
| Total | 9 | 4 | 3.52 | 4.62 | 18.77 | 1.06E+03 | 6.40E+02 | 2.42E+02 | 1.48E+02 | 5.70E+02 | 0.23 |
| work done | | | | | | | | | | | |
| (shear) | | | | | | | | | | | |
| Tender- | 9 | 13 | 3.33 | 4.06 | 18.8 | 2.15E+00 | −9.56E−01 | 4.16E−01 | −1.28E+00 | 9.71E−01 | 1.34 |
| ness | | | | | | | | | | | |
| (Adj.) | | | | | | | | | | | |
| pH 24 | 9 | 75 | 4.23 | 5.54 | 19.68 | 9.53E+00 | 5.01E+00 | 1.83E+00 | 4.61E+00 | 3.60E+00 | 0.92 |
| hours | | | | | | | | | | | |
| pH 45 | 9 | 75 | 3.72 | 4.71 | 14.07 | 2.26E+01 | 1.19E+01 | 4.37E+00 | 1.96E+00 | 8.58E+00 | 0.16 |
| minutes | | | | | | | | | | | |
| Pork | 9 | 105 | 3.91 | 5.03 | 14.3 | 1.37E+00 | 1.92E−01 | 2.36E−01 | −1.00E+00 | 3.97E−01 | −5.21 |
| flavour | | | | | | | | | | | |
| (Adj.) | | | | | | | | | | | |
| FG/FOG % | 9 | 121 | 7.59 | 10.7 | 19.78 | 2.20E+00 | −8.67E−01 | 3.23E−01 | 1.52E+00 | 5.50E−01 | −1.75 |
| SO % | 9 | 121 | 7.59 | 10.7 | 19.78 | 2.20E+00 | 8.67E−01 | 3.23E−01 | −1.52E+00 | 5.50E−01 | −1.75 |
| SO area | 9 | 121 | 7.13 | 10.03 | 18.68 | 5.40E+03 | 2.09E+03 | 7.97E+02 | −3.61E+03 | 1.36E+03 | −1.73 |
| FG/FOG | 9 | 123 | 4.73 | 6.35 | 11.53 | 8.56E+03 | −2.75E+03 | 1.23E+03 | 4.32E+03 | 2.04E+03 | −1.57 |
| Lean % | 9 | 126 | 3.78 | 4.86 | 8.68 | 2.27E+00 | 2.77E−01 | 3.08E−01 | −1.28E+00 | 4.84E−01 | −4.62 |

Notes to Table 5:
position is in relation to the first marker, add the position of the first marker for equivalence to USDA maps.
% var 1 = variance explained (reduction in residual) when QTL (a and d) are included in the model.
% var 2 = variance predicted from estimated a and d effects.
a = additive effect Du-LW, positive means a higher value in Du.
d = dominance effect, positive indicates a higher value, heterozygote is above the mean of the two homozygotes.
The results of the analysis for chromosome 5 are summarised in FIG. 2 for muscle fibre characteristics, tenderness and shear force. It shows that F values peak on chromosome 5 at positions 0 to 50 for shear force and around 70 for SO % and SO area. The estimates in Table 5 indicate that lower shear force values and lower SO % and area are associated with Duroc genes.
The results in FIG. 3, show high F values at the bottom of chromosome 9, for SO area and SO %, as well as FG/FOG area. As shown in Table 5, Duroc genes are associated with higher SO area and SO %, but lower FG/FOG area. Not shown in Table 5 is that lower shear forces are associated with Duroc genes in this region. At the top of chromosome 9, high F values are found for SO/cluster as well as peaks for shear force traits, indicating that in this case low SO/cluster and high

### EXAMPLE 2

QTL Analysis—Additional Animals

Following the initial whole genome scan described in Example 1 above, further animals recorded for the meat quality traits were genotyped by GeneSeek as described above for genetic markers on chromosome 5 and 9. The trait recording, genotyping and data analyses were carried out as described in Example 1. The results from the analysis of chromosome 5 and 9 for all the trait recorded animals—those described in Example 1 plus the additional 62 animals, i.e. a total of 180 are shown in Table 6.

Linkage analyses for chromosomes 5 and 9 are shown in the table below in which the published USDA map distances are compared from analysis of Phase 1 and Phase 2 data.

| Chromosome 5 | | | | Chromosome 9 | | | |
|---|---|---|---|---|---|---|---|
| | Con- | Phase | Phase | | Con- | Phase | Phase |
| Marker | sensus | 1 | 2 | Marker | sensus | 1 | 2 |
| SW413 | 0.0 | 0.0 | 0.0 | SWR68 | 0.0 | 0.0 | 0.0 |
| SW1482 | 32.0 | 24.4 | 24.6 | S0024 | 23.0 | 15.5 | 36.4 |
| SW439 | 66.0 | 62. 8 | 65.5 | SW827 | 49.0 | 46.9 | 79.3 |
| S0005 | 82.0 | 79.9 | 83.2 | SW727 | 72.0 | 77.0 | —¹ |
| SW904 | 103.0 | 90.5 | 103.7 | SW539 | 75.0 | 77.6 | —¹ |
| SWR1112 | 124.0 | 112.3 | —¹ | SW2093 | 100.0 | 97.8 | 125.9 |
| | | | | SW2116 | 126.0 | 129.6 | 155.1 |

¹Not included in phase 2

The results of the analysis for chromosome 5 are summarised in FIG. 4 for muscle fibre characteristics, tenderness and shear force (total work done). It shows that F values peak on chromosome 5 at positions 0 to 50 for shear force (total work done) and around 70 for SO % and SO area. The estimates in Table 6 indicate that lower shear force (total work done) values and lower SO % and area are associated with Duroc genes.

The results in FIG. 5, show high F values at the bottom of chromosome 9, for SO area and SO %. As shown in Table 6, Duroc genes are associated with higher SO area and SO %. Not shown in Table 5 is that lower shear forces (total work done) are associated with Duroc genes in this region. At the top of chromosome 9, high F values are found for SO/cluster as well as peaks for shear force traits, indicating that in this case low SO/cluster and high shear force (total work done) are associated with ‘Duroc’ genes (Table 6).

TABLE 6
Genome scan results by chromosome for the extended number of animals

| | | | F | % var | % var | Trait | | | | | Dominance |
|---|---|---|---|---|---|---|---|---|---|---|---|
| Trait | Chrom. | Position | ratio | 1 | 2 | s.d. | a | s.e. | d | s.e. | ratio |
| Sos/cluster | 5 | 0 | 2.83 | 1.94 | 3.9609 | 0.84 | 0.222 | 0.101 | −0.115 | 0.159 | −0.52 |
| Total work done | 5 | 15 | 4.83 | 4.89 | 13.631 | 1132.7 | −464.5 | 166 | −517.7 | 311.3 | 1.11 |
| Mean SO area | 5 | 65 | 5.09 | 4.23 | 8.1271 | 5878.6 | −2360.1 | 740.4 | −306.8 | 1144.5 | 0.13 |
| % SO | 5 | 65 | 5.19 | 4.53 | 8.3419 | 2.38 | −0.963 | 0.3 | −0.188 | 0.463 | 0.20 |
| SOs/cluster | 9 | 59 | 7.28 | 6.36 | 69.862 | 0.849 | −0.173 | 0.152 | 1.398 | 0.389 | −8.08 |
| Total work done | 9 | 78 | 4.39 | 4.36 | 28.237 | 1132.7 | 309.7 | 183.9 | 1121.3 | 430.8 | 3.62 |
| Mean SO area | 9 | 154 | 6.92 | 6.02 | 8.8864 | 5878.6 | 1767.4 | 619.9 | −2456.9 | 994.2 | −1.39 |
| % SO | 9 | 155 | 7.34 | 6.41 | 8.8266 | 2.38 | 0.727 | 0.244 | −0.971 | 0.384 | −1.34 |

Notes to Table 6:
position is in relation to the first marker, add the position of the first marker for equivalence to USDA maps.
% var 1 = variance explained (reduction in residual) when QTL (a and d) are included in the model.
% var 2 = variance predicted from estimated a and d effects.
a = additive effect Du-LW, positive means a higher value in Du.
d = dominance effect, positive indicates a
higher value, heterozygote is above the mean of the two homozygotes.

### REFERENCES

Barton-Gade (1989) The effect of breed on meat quality characteristics in pigs. 35th International congress of meat science and technology. Kulmbach.

Bejerholm (1984) Experience in taste testing pork at the Danish Meat Research Institute Proceedings of the 30th European Meeting of Meat Research workers.

Calkins et al (1981) Journal of Food Science 46: 708.

Carpenter et al (1996) Journal of Animal Science 74: 388-393.

Clutton-Brock (1981) Domesticated animals from early times London, Heinemann/British Museum.

Cockett et al (1994) Pro. Nat. Acad. Sci. USA 91: 3019-3023.

Gandemer and Legault (1990) Porc Magazine 222: 31-37.

Green et al (1990) Documentation for Cri-Map version 2.4. St. Louis, Washington University School of Medicine.

Handel and Stickland (1987) Journal of Anatomy 152: 107-119.

Haley et al (1994) Genetics 136:1195-1207.

Huston and Krebs (1968) Biochemistry 7: 2116.

Iwamoto et al (1983) Japanese Journal of Zootechnical Science 54(6): 392-400.

Koohmaraie et al (1995) Journal of Animal Science 73: 3596.

Lander and Kruglyak (1995). Nature Genetics 11: 241-247.

Maltin et al ( ) In press Animal Science.

Martel et al (1988) Journal of Animal Science 66: 41-46.

Mason (1988) World Dictionary of Livestock Breeds. Wallingford, Oxon, UK, C.A.B International.

Mellgren (1980) FEBS Letts 109: 129.

MLC (1992) Stotfold pig development unit—Second trial results, Meat and Livestock Commission, Milton Keynes.

MLC (1996) Pig Yearbook. Milton Keynes, Meat and Livestock Commission.

Ockerman et al (1984) Journal of Animal Science 59: 981.

Peter et al (1972) Biochemistry 11: 2627-2633.

Purchas et al (1990) New Zealand Journal of Agricultural Research 33: 97-104.

Ruusunen (1993) The fibre-type composition and capillary density in M L. Dorsi of different pig cross-breed. Pork Quality: Genetic and metabolic factors E. Puolanne, D. I. Demeyer, with, M. Ruusunen and S. Ellis. Wallingford, CAB International: 301.

Seideman and Crouse (1986) Meat Science 17: 55.

Seideman and Theer (1986) Journal of Food Quality 9: 251.

Skorjanc et al (1994) Zbornik biotehniske fakultete universe Ljubljani Kmetiistvo 64: 63-70.

Sorimachi et al (1993) J. Biol. Chem. 268: 10593.

Sorimachi et al (1989) J. Biol. Chem. 264: 20106.

Soumillion et al (1997) Mammalian Genome 8: 564.

Stumpe (1989) Zusammenhaenge zwischen sensorischen und technologischen Qualitaetseigenschaften des Schweinefleisches. Dissertation Institut fuer Tierzuchtwissenschaften, Bonn, B.R.D.

Szentkuti and Cassens (1978) “Die Verteilung der Fasertypen I, II A und II B im M Longissimus dorsi und M. semitendinosus von Schweinen verschiedenen Alters” Deutsche Tierarztliche Wochenschrift 85(1): 23-27.

Taylor et al (1994) J. Anim. Sci. 73: 1351.

Uhrin et al (1986) Zivocisna Vyroba 31(12):1065-1074.

Warkup et al. (1995) Consumer perceptions of texture; the most important quality attribute of meat? Expression of tissue proteinasese and regulation of protein degradation as related to meat quality A. Ouali, D. I. Demeyer and F. J. M. Smulders. Utrecht: ECCEAMST.

1. An assay to identify pigs with a genetic predisposition for improved meat quality, wherein said assay comprises:
a) obtaining a DNA sample from a test pig;
b) analysing the sample to determine the allelic variant(s) present at at least one genetic marker, wherein said marker is selected from:
i) SW413, SW1482, SW439, S0005, SW904 or regions of chromosome 5 spanning therebetween; or
ii) SWR68, S0024, SW827, SW727, SW539, or regions of chromosome 9 spanning therebetween; or
iii) SW2093, SW2116 or regions of chromosome 9 spanning therebetween; and
c) using the genotypic data from said marker(s) to select for pigs of the preferred genotype.

2. The assay of claim 1, wherein in step c) pigs with high meat quality traits are selected.

3. The assay as claimed in either one of claims 1 and 2 wherein said method comprises:
a) obtaining a DNA sample from said pig;
b) assaying said DNA sample for a sequence identical with or complementary to the genetic markers.

4. The assay as claimed in any one of claims 1 to 3 wherein the sample is analysed to determine the allelic variant(s) present at a genetic marker which is located:
i) on chromosome 5 in respect of shear force;
ii) between SW1482 and SW904 on chromosome 5 in respect of fitness traits; and/or
iii) between SWR68 and SW2093 on chromosome 9; and/or
iv) between SW2093 and SW2116 on chromosome 9;

5. The assay as claimed in any one of claims 1 to 4 wherein the sample is analysed to determine allelic variant(s) present at a genetic marker on chromosome 5 and at a genetic marker on chromosome 9.

6. The assay as claimed in any one of claims 1 to 5 wherein allelic variant(s) present at three or more distinct genetic loci are analysed.

7. The assay as claimed in any one of claims 1 to 6 which said genetic markers are selected from SW413, SW1482, SW439, S0005, SW904 or regions of chromosome 5 spanning therebetween.

8. The assay as claimed in any one of claims 1 to 6 which said genetic markers are selected from SWR68, S0024, SW827, SW727, SW539 or regions of chromosome 9 spanning therebetween.

9. The assay as claimed in any one of claims 1 to 6 which said genetic markers are selected from SW2093, SW2116 or regions of chromosome 9 spanning therebetween.

10. A method to identify pigs with a genetic predisposition for improved meat quality, wherein said method comprises:
a) obtaining DNA samples from a population of pigs;
b) genotyping at least a sample of said population for pre-determined markers that map within or close to the QTL for meat quality traits on chromosome 5 and 9 at a location displaying a high F ratio;
c) measuring meat quality traits for at least a sample of said population;
d) correlating the presence of allelic variants of said markers with said meat quality traits;
e) obtaining a DNA sample from a test pig;
f) analysing the sample to determine the allelic variant(s) present at a said selected genetic marker; and
g) using said marker results to select for pigs of the preferred genotype.

11. The method of claim 10, wherein said markers are derived from SW413, SW1482, SW439, S0005, SW904, SWR68, S0024, SW827, SW727, SW539, SW2093 or SW2116.

12. The method of claim 10, wherein said markers which map within the QTL for the meat quality traits of tenderness, shear force or muscle fibre traits.

13. The method of claim 10, wherein said markers are located between SW1482 and SW904 on chromosome 5, or between SWR68 and SW2093 on chromosome 9, or between SW2093 and SW2116 on chromosome 9.

14. The method as claimed in any one of claims 10 to 13, wherein genotypic data from more than one marker is analysed, and each marker allows the allelic variation at different QTL associated with separate meat quality traits to be predicted.

15. The method as claimed in claim 14, wherein genotypic data from at least three markers that each allow the allelic variation at different QTL associated with separate meat quality traits to be predicted are used in combination to select for pigs of the preferred genotype.

16. The method of any one of claims 10 to 15 wherein said genetic markers are selected using a method selected from the group consisting of microsatellites; restriction fragment length polymorphisms (RFLPs), single strand conformational polymorphisms (SSCP), double strand conformational polymorphisms, single nucleotide polymorphisms (SNPs), AFLP™ (amplified fragment length polymorphisms, DNA chips, variable number of tandem repeats (VNTRs, minisatellites), random amplified polymorphic DNA (RAPDs), heteroduplex analyses, and allele-specific oligonucleotides (ASOs).

17. The method of any one of claims 10 to 16, wherein said sample is selected from the group consisting of blood, semen (sperm), ascites and urine, liver tissue, muscle, skin, hair follicles, ear, tail, fat and testicular tissue.

18. A method of selecting pigs for use in breeding programs, said method comprising obtaining a DNA sample from a test pig and analysing said sample to determine the allelic variant(s) present at a genetic marker selected from:
i) SW413, SW1482, SW439, S0005, SW904 or regions of chromosome 5 spanning therebetween; or
ii) SWR68, S0024, SW827, SW727, SW539, or regions of chromosome 9 spanning therebetween; or
iii) SW2093, SW2116 or regions of chromosome 9 spanning therebetween; and
using the genotypic data from said marker to select for pigs having the required genotype.

19. A kit to identify a pig having a genetic disposition for high meat quality, said kit comprising at least three genetic markers having the ability to identify specific allelic variant(s) at three separate QTL indicative of meat quality.

20. A method of determining the genetic predisposition of a pig to yield meat of improved meat quality, said method comprising detecting genes located between the following pairs of markers:
i) SW413 and SW904 on chromosome 5;
ii) SWR68 and SW539 on chromosome 9; and
iii) SW2093 and SW2116 on chromosome 9;
wherein said genes are characterised by having allelic variant(s) which can influence meat quality or its component traits, or which are associated with variation in meat quality or its component traits.

21. The method as claimed in claim 20 wherein the genes are located between the positions of the genetic markers SW413 and SW904 on chromosome 5, and variation in said genes influence meat quality or its component traits.

22. The method as claimed in claim 20 wherein the genes are located between the positions of the genetic markers SWR68 and SW539 or between SW2093 and SW2116 on chromosome 9, and variation in said genes can influence meat quality or its component traits.

23. The method as claimed in claim 20 wherein the genes are located between the positions of the genetic markers SW413 and SW904 on chromosome 5, and variation in said genes associated with variation in meat quality or its component traits.

24. The method as claimed in claim 20 wherein the genes are located between the positions of the genetic markers SWR68 and SW539 or between SW2093 and SW2116 on chromosome 9, and variation in said genes are associated with variation in meat quality or its component traits.
